(19) United States
(12) Patent Application Publication
(10) Pub. No.: US 2025/0157615 A1
(43) Pub. Date: May 15, 2025

(54) METHOD, A SYSTEM AND A COMPUTER PROGRAM PRODUCT FOR INSTRUCTING ORAL HYGIENE

(71) Applicant: DentView Oy, Kempele (FI)

(72) Inventors: Mika Malinen, Kempele (FI); Eeva Malinen, Kempele (FI); Jouko Malinen, Oulu (FI)

(73) Assignee: DentView Oy, Kempele (FI)

(21) Appl. No.: 18/389,285

(22) Filed: Nov. 14, 2023

(51) Int. Cl. G16H 20/30; G16H 10/20; G16H 10/60; G16H 15/00

(52) U.S. Cl. CPC G16H 20/30; G16H 10/20; G16H 10/60; G16H 15/00

(57) ABSTRACT

A method for instructing oral hygiene for a customer is disclosed. In the method, the customer seeking oral hygiene instruction visits a health care facility where a professional working the health care facility prepares a personal dental status data of mouth of the customer. The personal dental status data is transferred to a customer's terminal device and includes an instruction application including a general dental map. The instruction application maps the personal dental status data to the general dental map for generating an instruction dental map. The instruction application presents an oral hygiene instruction session to the customer and the oral hygiene instruction session corresponds to the instruction dental map where areas needing attention are highlighted visually.

### BACKGROUND OF THE INVENTION

### Field of the Invention

The invention relates to a method and a system for instructing a customer in oral hygiene. The invention also relates to a computer program product for instructing a customer in oral hygiene on a customer's terminal device comprising at least one processor and at least one memory.

### Description of Related Art

Oral diseases pose a major health burden for many countries and affect people throughout their lifetime, causing pain, discomfort, disfigurement and even premature death. Treatment for oral health conditions is expensive and usually not part of universal health coverage (UHC). In most high-income countries, dental treatment averages 5% of total health expenditure and 20% of out-of-pocket health expenditure. The majority of oral health diseases such as dental caries and periodontitis are largely preventable and can be treated in their early stages. Reducing oral health conditions calls for a reform of oral health systems to shift the focus from invasive dental treatment to prevention and more minor treatment. It has been found that personal instructions significantly improve this. The burden of oral diseases can be reduced through instructions by addressing common risk factors. Oral self-care guidance is part of preventive work and includes patient motivation, teaching a healthy diet and lifestyle, analyzing one's own risks, and directing home care such as tooth-brushing.

It is a well-known fact that many people have improper habits when performing oral hygiene. This includes, for example, tooth-brushing. Oral self-care guidance is given in the dental clinics, but there are not enough resources for adequate preventive work. Preventive work can often also be targeted at specific groups of patients. In order to effectively prevent oral diseases, preventive work should be increased and targeted to all patient groups. However, this is quite time-consuming and people may not respond to the instructions. In order to transfer knowledge more effectively, it would be good to use self-learning as a form of teaching.

There are many methods in which dental hygiene is guided in different ways. Patent publication US20070074359 discloses a teaching toothbrush that communicates with a user. Patent publication U.S. Pat. No. 9,498,053 shows a functional toothbrush that collects data on the user's brushing habits and forwards them to an external device. Patent publication WO2009141489 presents a toothbrush monitoring device in the toothbrush. The device monitors the user's brushing habits and can guide the user. Data acquired by the monitoring device can be sent to a remote device and also received. What is common for these methods, is that they are quite complicated and instructions for users are vague and not particularly specific.

### BRIEF SUMMARY OF THE INVENTION

An object of the invention is a solution that can significantly reduce the disadvantages and drawbacks of the prior art. In particular, the object of the invention is a method that improves oral hygiene instruction at home.

The objects of the invention are attained that is characterised by what is stated in the independent patent claims. Some advantageous embodiments of the invention are disclosed in the dependent claims.

The invention is a method for instructing oral hygiene for a customer. In the method, the customer seeking oral hygiene instruction visits a health care facility where a professional working the health care facility prepares a personal dental status data of mouth of the customer. The personal dental status data is transferred to a customer's terminal device comprising an instruction application comprising a general dental map. The instruction application maps the personal dental status data to the general dental map for generating an instruction dental map. The instruction application presents an oral hygiene instruction session to the customer and the oral hygiene instruction session corresponds to the instruction dental map where areas needing attention are highlighted visually.

In one embodiment of the invention is a method for instructing oral hygiene. In one advantageous embodiment of the invention, the method comprises steps where a customer who is seeking oral hygiene instruction visits a health care facility. A professional in the health care facility prepares a personal dental status data of mouth of the customer, and the personal dental status data is transferred to a customer's terminal device comprising an instruction application. The instruction application comprises a general dental map. The instruction application maps the personal dental status data to the general dental map for generating an instruction dental map, and the instruction application presents an oral hygiene instruction session to the customer and the oral hygiene instruction session corresponds to the instruction dental map.

In one embodiment of the method, the customer is presented with a question set at the health care facility, and the customer's answers to the question set forms a part of the personal status data.

In a second embodiment of the method, the question set is stored in the customer's terminal device or an interactive device at the health care facility, and the used device stores the answers and links an identifier to the answers, and the professional uses the identifier to find the answers relating the customer.

In a third embodiment of the method, the personal dental status data prepared by the professional is linked to the identifier, and the transfer of the personal dental status data to customer's terminal device is initiated by the customer by using the identifier.

In a fourth embodiment of the method, the personal dental status data prepared by the professional is saved to a cloud service and the instruction application is configured to load the personal dental status data linked to the identifier.

In a fifth embodiment of the method, the identifier is linked to the answers and the personal dental status data for a pre-determined period of time.

In a sixth embodiment of the method, the general dental map corresponds to the customer's age.

In a seventh embodiment of the method, the instruction dental map comprises at least one visualised marking for dental areas needing attention.

In an eighth embodiment of the method, the oral hygiene instruction session comprises animated instructions relating visualised markings.

In a ninenth embodiment of the method, the personal dental status data comprises previous medical recordings.

In a tenth embodiment of the method, the personal dental status data in the terminal device is updated after the oral hygiene instruction session.

In an eleventh embodiment of the method, the personal dental status data prepared by the professional in the health care facility is updated using the customer's updated personal dental status data.

In one embodiment of the invention is a system for instructing a customer in oral hygiene. In one advantageous embodiment of the invention, the system comprises an interactive device, a terminal device, and an instruction application stored in the terminal device. The interactive device comprises an instruction arrangement, and the instruction arrangement comprises a processing unit, a memory, a display, and in the memory is stored a question set. The instruction arrangement is configured to present questions from the question set to the customer. The instruction arrangement is further configured to provide to the customer an identifier and load customer's answers to the questions to a cloud service, and the identifier is linked to the customer's answers. The identifier is configured to be presented or given to a dental professional who retrieves the answers using the identifier who retrieves the answers using the identifier, and a personal dental status data of mouth of the customer is prepared by the dental professional using the answers and medical examination of the customer's mouth. The personal dental status data is configured to be stored to the cloud service, and the personal dental status data is linked to the identifier. The instruction application is configured to load the personal dental status data to the terminal device using the identifier and choose a general dental map based on the personal dental status data and to map the personal dental status data to the general dental map for generating an instruction dental map. The instruction application is further configured to use the instruction dental map for presenting an oral hygiene instruction session to the customer.

In one embodiment of the system, the instruction dental map comprises at least one visualised marking for dental areas needing attention.

In one embodiment of the invention is a computer program product for instructing a customer in oral hygiene on a customer's terminal device comprising at least one processor and at least one memory. In one advantageous embodiment of the computer program product, the customer has an identifier that refers to a personal dental status data of mouth of the customer, and the personal dental status data and a set of general dental maps are stored in a cloud service. The computer program product is an instruction application comprising program instructions which, when stored in the memory and executed on the processor causes the terminal device to load the personal dental status data from the cloud service relating to the identifier and choose and load a general dental map based on the personal dental status data, and to map the personal dental status data to the general dental map for generating an instruction dental map, and the instruction dental map comprises at least one visualised marking for dental areas needing attention based on the personal dental status data. The terminal device is also caused to present an oral hygiene instruction session to the customer on the terminal device and the oral hygiene instruction session comprises the instruction dental map, and to repeat the oral hygiene instruction session to the customer a certain number of times and at intervals, both of which are pre-determined.

It is an advantage of the invention that it provides a method and a system that are particularly suitable for instructing a customer at home on how to maintain oral hygiene. Also, when using the invention, the customer may provide more truthful answers than he or she would to another person. Furthermore, the invention reduces the pressure on dental healthcare professionals to provide basic instruction, allowing them to focus on providing more advanced dental care. An advantange of the invention is that it provides a new way of teaching preventive self-care, reducing the occurrence of common oral diseases, such as caries and periodontitis, and shortening dental care queues.

It is a further advantage of the invention that it can be easily optimized for different customer segments.

The invention also provides a device that is interactive and it entertains and relaxes the customer.

The invention also makes it possible to gather data from a group of customers and use the data for improving the instruction programs and the question sets.

### BRIEF DESCRIPTION OF THE SEVERAL VIEWS OF THE DRAWINGS

In the following, the invention is described in detail. The description refers to the accompanying drawings, in which.

FIG. 1 depicts an example of a method for instructing oral hygiene as a flow chart according to an embodiment,

FIG. 2 depicts an example of a system for instructing oral hygiene according to an embodiment,

FIG. 3 depicts an example of an interactive device,

FIG. 4a depicts an example of a general dental map,

FIG. 4b depicts an example of an instruction dental map,

FIG. 5 depicts an example of an oral hygiene instruction session, and

FIG. 6 depicts an example of a customer's terminal device according to an embodiment.

### DETAILED DESCRIPTIONS OF THE INVENTION

The embodiments in the following description are given as examples only and someone skilled in the art can carry out the basic idea of the invention also in some other way than what is described in the description. Though the description may refer to a certain embodiment or embodiments in several places, this does not mean that the reference would be directed towards only one described embodiment or that the described characteristic would be usable only in one described embodiment. The individual characteristics of two or more embodiments may be combined and new embodiments of the invention may thus be provided.

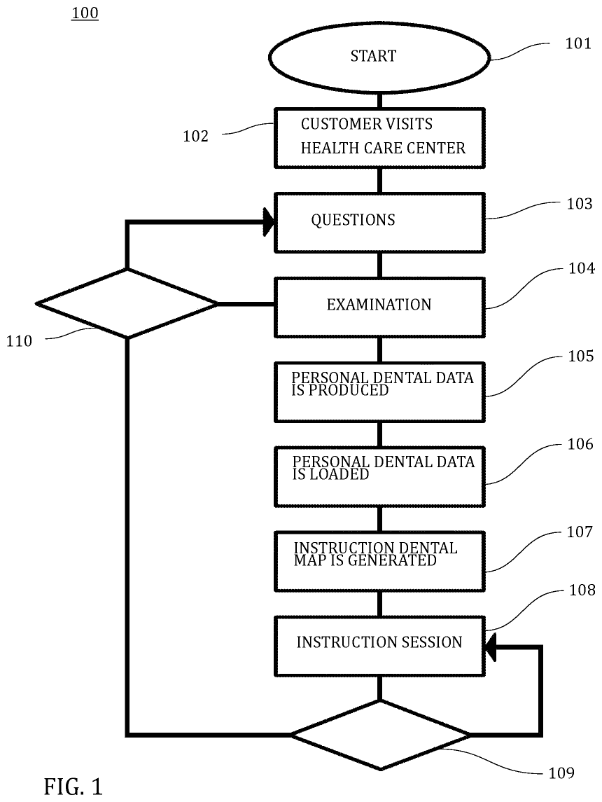

FIG. 1 shows an embodiment of a method for instructing oral hygiene 100 as a flow chart. This embodiment comprises following steps.

The method 100 is initiated at step 101 when a customer starts seeking oral hygiene instructions. The customer has a terminal device comprising an instruction application. At step 102 the customer visits a health care facility. The health care facility may be a dental clinic or a similar facility.

At step 103 the customer is presented with a question set at the health care facility. In some embodiments, the customer can choose between different question sets by answering questions during the questioning. In some embodiments, the contents can include a multitude of different question sets and only some of them are presented to the customer. The question sets are designed to resolve at least the customer's background information and current dental status. The question set is stored in the customer's terminal device or an interactive device at the health care facility, and the used device stores the customer's answers and links an identifier to said answers. In some embodiments, the customer's answers are stored to a cloud service. The identifier is given to the customer, or the customer may choose the identifier. The identifier is, for example, a QR-code, an image, or some other code.

At step 104 a professional in the health care facility meets the customer and checks the customer's dental status. In addition to a physical examination this may include some additional questions. The professional acquires the identifier from the customer to find the answers relating the customer. The professional prepares a personal dental status data of mouth of the customer based on the examination and the customer's answers at step 105. Also, in some embodiments, the personal dental status data comprises a plan for oral care compiled by the professional. In some embodiments, the professional includes previous medical recordings of the customer to the personal dental status data. In some embodiments, the personal dental status data comprises other health related information. These comprise, for example, risk factors such as smoking.

At step 106 the personal dental status data is transferred to the customer's terminal device comprising the instruction application. In some embodiments, the transfer of the personal dental status data to customer's terminal device is initiated by the customer by using the identifier. In some embodiments, the personal dental status data prepared by the professional is saved to a cloud service and the instruction application is configured to load the personal dental status data linked to the identifier. In some embodiments, the identifier is linked to the answers and the personal dental status data for a pre-determined period of time. In some embodiments, after that period of time the identifier is free to be used again. This embodiment may be used if the identifier is an image or a figure. Also, at the step 106, a general dental map is loaded to the customer's terminal. The general dental map describes a general mouth image containing at least teeth and gums. In some embodiments, the general dental map corresponds to the customer's age, and the general dental map is chosen based by the customer's answers or other information. In some embodiments, the instruction application comprises the general dental map or a set of general dental maps.

At step 107 the instruction application maps the personal dental status data to the general dental map for generating an instruction dental map. The instruction dental map comprises at least one visualised marking for dental areas needing attention. Said dental areas are based on the personal dental status data and more specifically to the examination of the customer's mouth.

At step 108 the instruction application presents an oral hygiene instruction session to the customer and the oral hygiene instruction session corresponds to the instruction dental map. The instruction dental map comprises at least one visualised marking for dental areas needing attention. The oral hygiene instruction session is configured to give instructions to the customer for dental care. These instructions include instructions for maintaining oral hygiene and also point out what are the places the customer should pay special attention or if a specific technique is needed, for example such as flossing. In some embodiments, the oral hygiene instruction session comprises animated instructions relating visualised markings placed on the instruction dental map. In some embodiments, the instruction dental map is 3D-animation and the customer or the instruction application may rotate or otherwise move the instruction dental map during the oral hygiene instruction session for highlighting the dental areas needing special attention. In some embodiments, the oral hygiene instruction session includes other instructions based on the personal dental status data.

In some embodiments, the personal dental status data in the terminal device is updated after the oral hygiene instruction session. In some embodiments, the updated personal dental status data is send to the cloud service. In some embodiments, the updated personal dental status data in the terminal device is used to update information related to the customer when the customer is visiting the professional at the health care facility.

At step 109 is checked if the oral hygiene instruction session should be repeated. In some embodiments, the oral hygiene instruction session is configured to be repeated to the customer a certain number of times and at intervals, both of which are pre-determined. The oral hygiene instruction session is repeated at the step 108. After some predetermined interval or when some date has been reached, the instruction application may recommend the customer visit the health care facility. At step 110 it is checked if the customer should repeat the question set again at the step 103 or go to straight the step 104 for meeting the professional in the health care facility. The professional examines the customer and update the personal dental status data if needed.

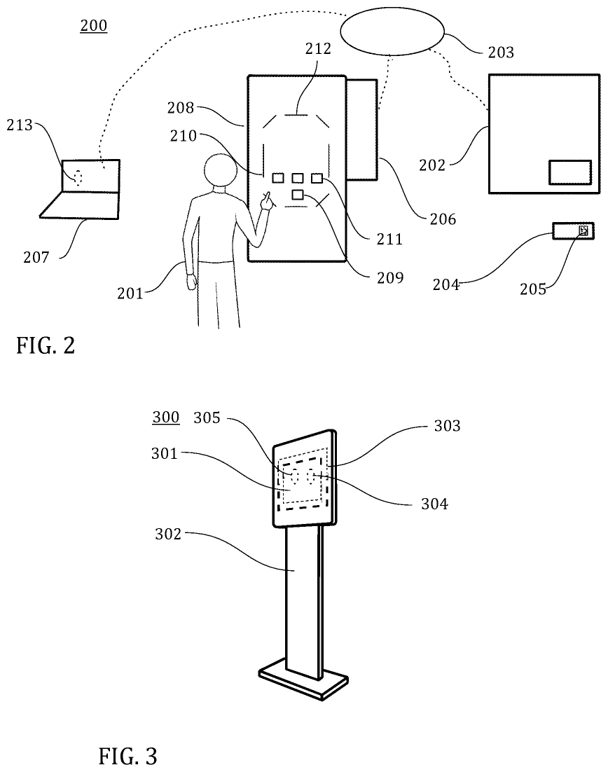

FIG. 2 shows an embodiment of a system 200 for instructing a customer 201 on how to maintain oral hygiene, i.e. providing an oral hygiene instruction session. The system is partly situated, for example, at a dental clinic or a similar health care facility. The system comprises an interactive device 208, a terminal device 204, a cloud service 203, and an instruction application 205 in the terminal device. In this example, the system further comprises a computational device 207 used by a dental professional working in the health care facility and a database 202 containing medical recordings of the customer.

The interactive device 208 is, for example, an information kiosk, a computer, or a port-able device. The interactive device comprises an instruction arrangement 212. The instruction arrangement comprises a processing unit, a memory, a touchscreen display and a communication arrangement 206 for communicating with the cloud service 203. A question set is stored in the memory and a set of guide path programs. During an initial instruction session the instruction arrangement is configured to present the customer with questions 210 from the question set and select a guide path program the set of guide path programs for the customer based on the customer's answers. The device is further configured to provide an identifier 209 to the customer. The identifier relates to the answers the customer has given. The identifier is, for example, an image or some similar entity that is distinguished from other identifiers.

The instruction arrangement 212 is configured to load customer's 201 answers to the questions 210 to the cloud service 203 using the communication arrangement 206. In some embodiments, the instruction arrangement is installed in the customer's terminal device 204, i.e. the terminal device is the interactive device 208. The identifier 209 is configured to be presented or given to a dental professional who retrieves the answers using the identifier and the professional dental application 213 in the computational device 207. The dental professional performs an oral examination. Results of the oral examination and the answers are used by the dental professional for producing a personal dental status comprising at least data of mouth of the customer. The professional dental application combines these as a personal dental status data. This means that the personal dental status data comprises both current oral medical status and information of the customer 201. In some embodiments, the dental professional uses also previous medical recordings of the user by contacting the database 202. The database is, for example, a national health database or similar database containing recordings such as the customer's dental charts. The personal dental status describes current status of customer's oral health. It includes dental areas needing attention such as areas containing incipient caries or areas containing gingivitis.

The personal dental status data is stored to the cloud service 203, and the personal dental status data is linked to the identifier 209. The identifier may be same as the customer was given after answering the questions, but the customer may be provided a new one after the personal dental status data is generated. The instruction application 205 is configured to load the personal dental status data to the terminal device 204 using the identifier. In some embodiments, this is done by the customer 201 by choosing the identifier when using the instruction application 205 in the terminal device. In some embodiments, the identifier is linked to the answers and the personal dental status data for a pre-determined period of time, i.e. the identifier is valid only a certain time period and after that it is expired. The instruction application is configured to load the personal dental status data to the terminal device using the identifier and choose a general dental map based on the personal dental status data. The general dental map describes a standard dental chart. In some embodiments, there is a set of general dental maps, and each general dental map corresponds to a standard feature such as age of a customer. The general dental map is downloaded to the terminal device with the personal dental status or the instruction application contains the personal dental status or set of them. The instruction application then maps the personal dental status data to the general dental map for generating an instruction dental map. This means that the instruction application combines at least part of the personal dental status data and the general dental map. The instruction dental map is a dental map comprising at least one dental area needing attention or special care.

The instruction application 205 is further configured to use the instruction dental map for presenting an oral hygiene instruction session to the customer 201. The oral hygiene instruction session comprises at least highlighted areas in the instruction dental map for pointing out to the customer 201 where attention or special care should be aimed. The instruction dental map may be 3-D-animated or it contain animated parts. The oral hygiene instruction session comprises instructions, for example animations, how to tend areas needing special attention. Of course, the oral hygiene instruction session may include other instructions besides the instruction dental map.

The instruction application 205 is a computer program that is stored, for example, in the cloud service 203. The cloud service is configured to send the instruction application to the customer's 201 terminal device 204 when the terminal device sends a request. In some embodiments, the request is activated by reading a QR-code from the interactive device 208. Naturally, the instruction application can be acquired in numerous ways. The instruction application is configured to present an oral hygiene instruction session to the customer 201 on the terminal device 204, and the oral hygiene instruction session corresponds to the personal dental status data. The instruction application is configured to repeat the oral hygiene instruction session to the customer a certain number of times and at intervals that are both pre-determined. The instruction application, for example, is activated every evening during the instruction period when the customer brushes his or her teeth. In some embodiments, the customer activates the instruction application. Here, the pre-determined intervals means that the presented instruction sessions are counted until some pre-determined time period has passed between completed sessions. The customer may activate the instruction session as many times as he or she wishes, but the sessions are counted towards to a pre-determined amount of sessions only after the pre-determined time has passed between sessions. Also, in some embodiments, when there are different instruction sessions, the content of instruction session changes when a pre-determined time period has passed or the count of completed instruction sessions has reached some pre-determined number.

FIG. 3 shows an embodiment of an interactive device 300. In this embodiment, the interactive device is a part of an information kiosk for instructing a customer on how to maintain oral hygiene. The information kiosk is, for example, positioned at some health care facility. When the interactive device is activated, it will start an initial instruction session or any instruction session for the benefit of the customer. The initial instruction session serves to determine what type of instruction the customer needs. The initial instruction session includes a question set presented to the customer and the customer's answers are used to select a guide path program for the customer. The guide path program is selected from a set of guide path programs. The guide path programs are tailored for different customer groups. The initial instruction session may include some instructions or the contents of the instruction session are derived from the selected guide path program. It must be noted that some additional information about the customer can also be gathered during the instruction session.

The interactive device 300 comprises a mirror 301, a frame 302, and a touchscreen display 303. The touchscreen display is located behind the mirror. Of course, it is possible to use a conventional display. The device further comprises an instruction arrangement, and the instruction arrangement comprises a processing unit 304 and a memory 305. The display is used by the device and the instruction arrangement.

The touchscreen display 303 is a display that is able to render still images, animation and recorded video. The touchscreen makes it possible to implement an interface that uses touch-activated buttons. The processing unit controls the touchscreen display and other functions of the interactive device 300 and the instruction arrangement. The processing unit 304 is a computer or a similar arrangement having computing properties. The processing unit comprises at least a processor and persistent data storage. The processing unit is configured to activate an area of the touchscreen display. This activated area can consist of the whole area of the touchscreen display or part of it (as in several distinct areas of the touchscreen display). The memory of the processing unit contains at least a part of the instructions for maintaining oral hygiene that the interactive device 300 is presenting to the customer during the instruction session. The processing unit is configured to store the customer's responses and answers to the instructions and questions respectively. These responses may be interactions with the interface at some stage of the instruction session. In some embodiments, the responses may be recognized by the instruction device. In those embodiments, the instruction device comprises sensors to capture some of the customer's motions. The customer is provided an identifier that corresponds to the answers the customer has given. The identifier is further used by a dental professional for acquiring the customer's answer for generating a personal dental status.

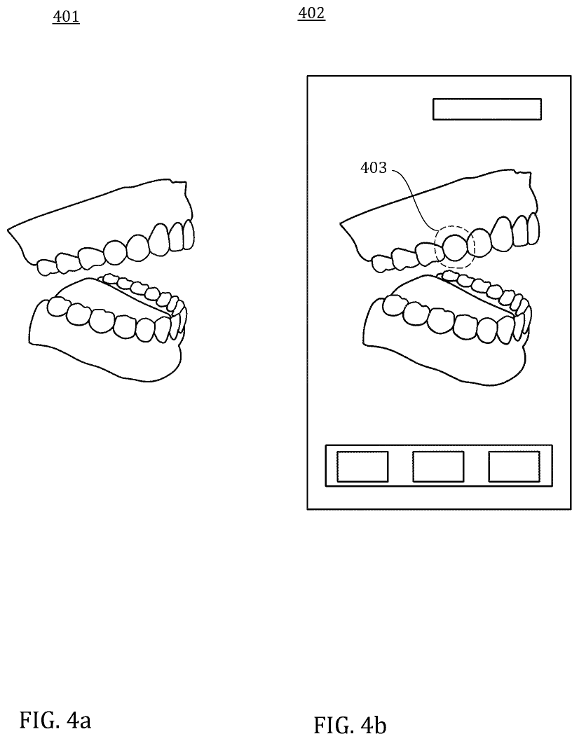

FIG. 4a shows an example of a general dental map 401 and FIG. 4b shows an example of an instruction dental map 402. The general dental map discloses a standard model dental map that is suitable for a customer. For example, the general dental map is chosen from a set of general dental maps based on age of the customer. A personal dental status of the customer is based on a set of questions asked from the customer and a medical examination of the customer made by a dental professional. The personal dental status comprises oral areas demanding attention that the dental professional has found. Said areas may need a special care such as intensive teeth brushing or flossing or similar operations the customer may perform. The instruction dental map is formed by an instruction application combining the information the personal dental status to the general dental map. Because the general dental map is not an exact model of the customer's mouth i.e. dental area, the instruction dental map is not either exact. However, the instruction dental map works very well when guiding the customer to the areas demanding special care. In FIG. 4b is marked an area 403, a tooth, where is incipient caries. The instruction application presents an oral hygiene instruction session where the customer is guided to brush said area particularly well.

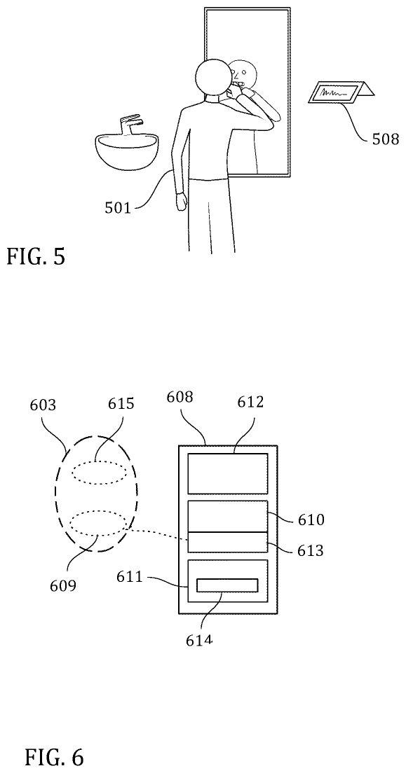

FIG. 5 shows an example of an oral hygiene instruction session at the customer's 501 home as presented by an instruction application that is loaded to a customer's terminal device 508 from a cloud service. The terminal device comprises a processor, a memory and a screen. The terminal device is, for example, a smartphone or a tablet. The instruction application shows images, animations or video clips on the screen.

In this example, the instruction session guides the customer 501 how to brush his or her teeth. The instruction session uses an instruction dental map for this. The instruction application is configured to issue a reminder to the customer about the scheduled instruction session. The customer activates the instruction application when he or she is ready to begin the instruction session. In this case, the customer should be at an appropriate place and have the necessary accessories at his or her disposal. In this example, the customer is in a bathroom in front of a mirror and the terminal device 508 is positioned in such a way that the customer is able to see the screen. The instruction application renders still images, animations or video clips on the screen that guide the customer towards efficient tooth-brushing using an instruction dental map. In addition to the visual instructions, the instruction application may use audible guidance in instructing the customer. These instruction sessions for promoting oral hygiene are repeated a certain number of times and at intervals that are both pre-determined. The contents of the instruction session and their schedules correspond to the selected guide path program and a personal dental status data.

In some embodiments, the instruction application comprises a performance recording phase for storing the customers response and actions to the instructions presented in the oral hygiene instruction session. These recordings can be, for example, audiovisual recordings or other feedback from the customer. The instruction application may comprise a questionnaire to this effect, which is displayed at the end of the instruction session. In some embodiments, the customer's responses are sent by the instruction application to an external service, such as a cloud service, that stores the responses and combined them with other customers' responses, and the combined results are used to develop the guide path programs and the oral hygiene instruction session further.

In some embodiments, the instruction application presents an evaluation session to the customer after a pre-determined period of time has passed from the last oral hygiene instruction session, and the evaluation session checks if the customer has made changes to his or her oral hygiene routines. In some embodiments, the instruction application issues a certificate for the customer after the last oral hygiene instruction session. This certificate may entitle the holder to a prize at the dental clinic where he or she attended the initial instruction session.

FIG. 6 shows an embodiment of a customer's terminal device 608 and an instruction application. The terminal device comprises a processor of the terminal device 610, a memory of the terminal device 611, a screen of the terminal device 612, and a connection arrangement of the terminal device 613. The instruction application is a computer program product. The instruction application comprises program instructions 614 which, when executed on a terminal device, cause the terminal device to present an oral hygiene instruction session to the customer. The oral hygiene instruction session comprises at least some visual instructions on the screen of the terminal device. These visual instructions are generated by using an instruction dental map.

The customer receives an identifier after he or she has passed an initial instruction session at a dental clinic or similar venue using an interactive device comprising an instruction arrangement. The identifier is, for example, a QR-code or an image. The initial instruction session chooses for the customer a guide path program from a set of the guide path programs. The identifier corresponds to the guide path program chosen to the customer. The guide path program determines which type and how many oral hygiene instruction sessions are to be presented to the customer. The instruction application can be downloaded from a cloud service 603 to the terminal device 608 and stored in the memory. The instruction application is configured to download a personal dental status data 615 and a general dental map 609 from the cloud service and produce an instruction dental map for the oral hygiene instruction session.

The instruction application repeats the oral hygiene instruction session a certain number of times and at intervals that are both pre-determined. In some embodiments, the contents of the given oral hygiene instruction session may change from session to session. The instruction application alerts the customer when the oral hygiene instruction session should be started. The customer activates the instruction application when he or she is ready. The instruction application determines how many times the oral hygiene instruction sessions has been presented to the customer and, if necessary, adjusts the new oral hygiene instruction session accordingly. The instruction application records the customer's progress and responses to the oral hygiene instruction session. In some embodiments, this information is actively gathered using the cam-era or microphone on the terminal device. In some embodiments, information is gathered pas-sively based on the customer's feedback. The instruction application may present some questions to enable this. In some embodiments, information is gathered in both ways. The instruction application stores this information in the memory of the terminal device 611 and uses the connection arrangement of the terminal device 613 to transmit at least some of the information to the cloud service 603. In some embodiments, the transmitting of the data is executed in real time. The transmitted information is stored in a database, which contains information from a plethora of customers. In some embodiments, the database contains only general information about customers and it omits any identifying cues. In some embodiments, the customers are identified, and the customer identifying information is stored with the customer responses. The identifying data comprise, for example, a time stamp, a phone number or other customer identifying data.

When the last oral hygiene instruction session is presented to the customer in accordance with the session schedule, the instruction application presents a progress summary to the customer. The summary is based on the stored customer responses. In some embodiments, the customer receives a certificate from the instruction application. In some embodiments, the customer receives a voucher that can be redeemed for a prize at the place where he or she attended the initial instruction session.

Some advantageous embodiments of the method and the system according to the invention have been described above. The invention is however not limited to the embodiments described above, but the inventive idea can be applied in numerous ways within the scope of the claims.

What is claimed is:

1. A method for instructing oral hygiene to a person, the method comprising steps of:
preparing personal dental status data of a mouth of the person;
transferring the personal dental status data to a terminal device, the terminal device comprising an instruction application comprising a general dental map and configured to generate a mapping of the personal data to the general dental map;
generating, via the instruction application, an instruction dental map; and
presenting, via the instruction application, an oral hygiene instruction session to the person; and
wherein the oral hygiene instruction session corresponds to the instruction dental map.

2. The method according to claim 1, further comprising the steps of:
presenting the person with a question set; and
wherein the personal dental status data comprises answers provided by the person to the question set.

3. The method according to claim 2, further comprising the steps of:
storing the question set in the terminal device, and
linking an identifier to the answers.

4. The method according to claim 3, further comprising the steps of:
linking the personal dental status data to the identifier, and
transferring the personal dental status data to the terminal device by customer initiation based upon the identifier.

5. The method according to claim 4, further comprising the steps of:
saving the personal dental status data to a cloud service; and
configuring the instruction application to load the personal dental status data linked to the identifier.

6. The method according to claim 3, further comprising the step of linking the answers and the personal dental status data for a pre-determined period of time.

7. The method according to claim 1, wherein the general dental map corresponds to an age of the person.

8. The method according to claim 1, wherein the instruction dental map comprises at least one visualised marking identifying dental areas requiring medical attention.

9. The method according to claim 8, wherein the oral hygiene instruction session comprises animated instructions relating visualised markings.

10. The method according to claim 1, wherein the personal dental status data comprises medical recordings.

11. The method according to claim 1, further comprising the step of updating the personal dental status data in the terminal after an oral hygiene instruction session.

12. The method according to claim 11, further comprising the step of updating the personal dental status data using personal dental status data.

13. A system for instructing a person on oral hygiene, the system comprising:
an interactive device,
a terminal device,
an instruction application stored in the terminal device, and
wherein the interactive device comprises an instruction arrangement comprising a processing unit, a memory, a display,
wherein a question set stored in the memory,
wherein the instruction arrangement is configured to:
present questions from the question set to the person,
present an identifier to the person, and
load answers to questions to a cloud service,
wherein the identifier is linked to the answers and configured to be retrievable, and
wherein a personal dental status data of a mouth of the person is:
prepared using the answers and a medical examination,
configured to be stored to the cloud service, and
linked to the identifier, and
the instruction application is configured to:
load the personal dental status data to the terminal device using the identifier;
choose a general dental map based on the personal dental status data; and
map the personal dental status data to the general dental map for generating an instruction dental map; and
the instruction application is further configured to use the instruction dental map for presenting an oral hygiene instruction session to the person.

14. The system according to claim 13, wherein the instruction dental map comprises at least one visualised marking for dental areas needing attention.

15. A non-transitory computer readable medium having stored thereon a set of instructions for carrying out the computer implemented method of instructing a customer in oral hygiene on a customer's terminal device, the method comprising the steps of:
loading personal dental status data relating to an identifier and choosing and loading a general dental map based on the personal dental status data;
mapping the personal dental status data to the general dental map for generating an instruction dental map, wherein the instruction dental map comprises at least one visualised marking for dental areas needing attention based on the personal dental status data;
presenting an oral hygiene instruction session on the terminal device, wherein the oral hygiene instruction session comprises the instruction dental map; and
repeating the oral hygiene instruction session a number of predetermined times and intervals; and
wherein the terminal device comprises at least one processor and at least one memory; and
wherein the customer includes an identifier comprising personal dental status data related to the mouth of the customer.
